(19) United States
(12) Patent Application Publication
(10) Pub. No.: US 2024/0271103 A1
(43) Pub. Date: Aug. 15, 2024

(54) GENETICALLY MODIFIED PORCINE CELLS, TISSUE, AND ANIMALS WITH REDUCED HUMAN XENOREACTIVITY AND METHODS OF USING THE SAME

(71) Applicant: THE TRUSTEES OF INDIANA UNIVERSITY, Bloomington, IN (US)

(72) Inventors: Ping LI, Carmel, IN (US); Burcin EKSER, Indianapolis, IN (US)

(21) Appl. No.: 18/568,607

(22) PCT Filed: Jun. 10, 2022
(86) PCT No.: PCT/US22/33004

§ 371 Date: Dec. 8, 2023

Related U.S. Application Data
(60) Provisional application No. 63/208,997, filed on Jun. 10, 2021.

(51) Int. Cl. C12N 5/071

(52) U.S. Cl. CPC C12N 5/069; C12N 2502/1164; C12N 2503/00; C12N 2510/04

(57) ABSTRACT

Disclosed are genetically modified porcine cells, tissues, organoids, and animals and methods of using the same for transplantation, and testing for xenoreactivity.

### CROSS-REFERENCE TO RELATED APPLICATIONS

This application claims the benefit of priority under 35 U.S.C. § 119(e) of U.S. Provisional Application No. 63/208,997, filed Jun. 10, 2021, the content of which is incorporated herein by reference in its entirety.

### STATEMENT REGARDING FEDERALLY SPONSORED RESEARCH OR DEVELOPMENT

This invention was made with government support under AI164002 awarded by the National Institutes of Health. The Government has certain rights in the invention.

### SEQUENCE LISTING

A Sequence Listing accompanies this application and is submitted as an ASCII text file of the sequence listing named “144578_00309_ST25.txt” which is 33,432 bytes in size and was created on Jun. 2, 2022. The sequence listing is electronically submitted via EFS-Web with the application and is incorporated herein by reference in its entirety.

### FIELD

The present disclosure relates to genetically modified porcine cells, tissues, organs, and animals having reduced antigenicity and that can be used, for example, in xenotransplantation.

### BACKGROUND

Pig-to-human xenotransplantation (XTx) offers a promising solution to address the persistent organ shortage for clinical transplantation¹. However, incompatibilities between pig and human species result in destructive human immune responses and ultimate failure of pig tissue/organ grafts. Genetic modification of pigs is an essential approach to overcome these obstacles². Elimination of all three known carbohydrate xenoantigens (αGal, Neu5Gc, and Sda), expression of human complement-regulatory proteins (e.g., CD46, CD55) and human coagulation-regulatory proteins (e.g., thrombomodulin, endothelial cell protein C receptor), expression of the anti-inflammatory molecule human heme oxygenase-1 (HO-1), and the introduction of the human macrophage-inhibitory ligand (CD47), brings XTx closer to reality. Recently, breakthrough progress has been made in preclinical XTx which has resulted in extended survival of pig-to-nonhuman primate (NIP) kidney XTx for more than a year^{4,5} (rhesus macaque), orthotopic heart XTx for more than 6 months⁶ (baboon), and heterotopic heart XTx more than 3 years⁷ (baboon).

Advances in gene-editing tools have made the engineering of porcine cells feasible and have accelerated the production of genetically-modified (GM) pigs. Multiple xenoantigens can be simultaneously eliminated in a single-step transfection⁸⁻¹⁰. Yet, the challenge remains on how to efficiently evaluate the human immune response induced by various genetic modifications, and to identify an ideal genetic combination. Currently, pig-to-NIP XTx is the standard preclinical model, which monitors NHP recipient survival (as surrogates for humans) after receiving life-supporting pig tissue/organ transplants. Pig-to-NHP XTx is a long process involving generation of GM pigs, XTx surgeries, and post-transplant animal care. It may take more than a year to complete a study, which is not ideal for the timely application of XTx. Moreover, the differences between human and NHPs in antibody reactivity to cells from various GM pigs suggest the potential limitation of NHP models for examining specific genetic modification(s).

Our previous studies demonstrated that the elimination of three carbohydrate xenoantigenic epitopes (αGal, N-glycolylneuraminic acid [Neu5Gc], and Sda) through disruption of the α1,3-galactosyltransferase gene (GGTA1), cytidine monophospho-N-acetylneuraminic acid hydroxylase gene (CMAH), and β-1,4-N-acetyl-galactosaminyltransferase 2 (β4galNT2) genes, dramatically reduces human antibody binding to porcine cells^{9,11}. The triple-gene knockout (TKO, GGTA1, CMAH, β4galNT2-knockout) pig is regarded as an essential genetic background for clinical XTx trials. However, elimination of porcine Neu5Gc may have detrimental effects in pig-to-NHP preclinical XTx trials. Both pigs and NHPs have CMAH gene encoding an enzyme that hydroxylates N-acetylneuraminic acid (Neu5Ac) to produce Neu5Gc.

Inactivation of the porcine CMAH gene results in Neu5Ac exposure, which most likely becomes the target of NHPs pre-existing antibodies and triggers antibody-mediated rejection in NHPs. Li et al¹³ reported that baboons have elevated antibody binding to TKO pig red blood cells (RBC) compared to the RBC from GGTA1/β4galNT2 double-knockout (DKO) pigs. A recent study of pig-to-baboon renal XTx revealed that the median survival of TKO pig kidneys (4 days) was significantly shorter than the survival of GGTA1-knockout (Gal-knockout, GTKO) kidneys (136 days)¹⁴. Antibody-mediated xenograft rejection is associated with CMAH gene disruption. Therefore, although the manipulation of the CMAH gene is required in clinical XTx trials, it appears to be detrimental in NHPs preclinical XTx trials. Other approaches to study the human immune response to GM pigs, such as ex vivo perfusion of a GM pig organ with human blood¹⁵ and in vitro analysis of the human immune response to PBMC or kidney fibroblasts from GM pigs^{10,11,16}, still requires the physical production of GM pig(s).

There is an urgent need to examine human humoral and cellular immune responses to GM porcine cells to efficiently identify suitable genetic modifications for XTx.

### SUMMARY

Disclosed herein are genetically modified porcine cells, tissues, and animals and methods of using the same for testing for xenoreactivity to a host immune system.

In a first aspect of the current disclosure, genetically modified porcine cells are provided. In some embodiments, the porcine cells comprise a knock-out mutation in each of genes GGTA, CMAH, b4galNT2, SLA-1, and B2M. In some embodiments, the porcine cell is a porcine liver-derived endothelial cell (pLDEC). In some embodiments, the porcine cell expresses CD31 and von Willebrand factor (VWF). In some embodiments, the porcine cell exhibits reduced immunoreactivity to a human serum sample compared to a genetically modified porcine cell comprising knock-out mutations only in genes GGTA, CMAH, and b4galNT2. In some embodiments, the porcine cell comprises one or more immortalization factor selected from SV40 T antigen, human telomerase (hTERT), simian virus 40 large T antigen, papillomavirus E6, papillomavirus E7, adenovirus E1A, Epstein-Barr virus, and human T-cell leukemia virus.

In another aspect of the current disclosure, organoids are provided. In some embodiments the organoids comprise a porcine cell with knock-out mutation in each of genes GGTA, CMAH, b4galNT2, SLA-1, and B2M. In some embodiments, the porcine cell is a porcine liver-derived endothelial cell (pLDEC). In some embodiments, the porcine cell expresses CD31 and von Willebrand factor (VWF). In some embodiments, the porcine cell exhibits reduced immunoreactivity to a human serum sample compared to a genetically modified porcine cell comprising knock-out mutations only in genes GGTA, CMAH, and b4galNT2. In some embodiments, the porcine cell comprises one or more immortalization factor selected from SV40 T antigen, human telomerase (hTERT), simian virus 40 large T antigen, papillomavirus E6, papillomavirus E7, adenovirus E1A, Epstein-Barr virus, and human T-cell leukemia virus.

In another aspect of the current disclosure, porcine tissue is provided. In some embodiments, the porcine tissue comprises a porcine cell with knock-out mutation in each of genes GGTA, CMAH, b4galNT2, SLA-1, and B2M. In some embodiments, the porcine cell is a porcine liver-derived endothelial cell (pLDEC). In some embodiments, the porcine cell expresses CD31 and von Willebrand factor (VWF). In some embodiments, the porcine cell exhibits reduced immunoreactivity to a human serum sample compared to a genetically modified porcine cell comprising knock-out mutations only in genes GGTA, CMAH, and b4galNT2. In some embodiments, the porcine cell comprises one or more immortalization factor selected from SV40 T antigen, human telomerase (hTERT), simian virus 40 large T antigen, papillomavirus E6, papillomavirus E7, adenovirus E1A, Epstein-Barr virus, and human T-cell leukemia virus.

In another aspect of the current disclosure, pigs are provided. In some embodiments, the pigs comprise a porcine cell with knock-out mutation in each of genes GGTA, CMAH, b4galNT2, SLA-1, and B2M. In some embodiments, the porcine cell is a porcine liver-derived endothelial cell (pLDEC). In some embodiments, the porcine cell expresses CD31 and von Willebrand factor (VWF). In some embodiments, the porcine cell exhibits reduced immunoreactivity to a human serum sample compared to a genetically modified porcine cell comprising knock-out mutations only in genes GGTA, CMAH, and b4galNT2. In some embodiments, the porcine cell comprises one or more immortalization factor selected from SV40 T antigen, human telomerase (hTERT), simian virus 40 large T antigen, papillomavirus E6, papillomavirus E7, adenovirus E1A, Epstein-Barr virus, and human T-cell leukemia virus.

In another aspect of the current disclosure, in vitro cultured porcine tissue is provided. In some embodiments, the in vitro cultured porcine tissue comprises porcine cells with a knock-out mutation in each of genes GGTA, CMAH, b4galNT2, SLA-1, and B2M, optionally, wherein the tissue is suitable for transplantation. In some embodiments, the porcine cells are porcine liver-derived endothelial cell (pLDEC). In some embodiments, the tissue exhibits reduced immunoreactivity to a human serum sample, compared to in vitro cultured porcine tissue comprising cells with knock-out mutations only in GGTA, CMAH, and b4galNT2.

In another aspect of the current disclosure, animals are provided. In some embodiments, the animals comprise a porcine cell with knock-out mutation in each of genes GGTA, CMAH, b4galNT2, SLA-1, and B2M. In some embodiments, the porcine cell is a porcine liver-derived endothelial cell (pLDEC). In some embodiments, the porcine cell expresses CD31 and von Willebrand factor (VWF). In some embodiments, the porcine cell exhibits reduced immunoreactivity to a human serum sample compared to a genetically modified porcine cell comprising knock-out mutations only in genes GGTA, CMAH, and b4galNT2. In some embodiments, the porcine cell comprises one or more immortalization factor selected from SV40 T antigen, human telomerase (hTERT), simian virus 40 large T antigen, papillomavirus E6, papillomavirus E7, adenovirus E1A, Epstein-Barr virus, and human T-cell leukemia virus.

In another aspect of the current disclosure, further animals are provided. In some embodiments, the animals comprise in vitro cultured porcine tissue comprising porcine cells with a knock-out mutation in each of genes GGTA, CMAH, b4galNT2, SLA-1, and B2M, optionally, wherein the tissue is suitable for transplantation. In some embodiments, the porcine cells are porcine liver-derived endothelial cell (pLDEC). In some embodiments, the tissue exhibits reduced immunoreactivity to a human serum sample, compared to in vitro cultured porcine tissue comprising cells with knock-out mutations only in GGTA, CMAH, and b4galNT2.

In another aspect of the current disclosure, methods of testing genetically modified porcine cells for xenoreactivity are provided. In some embodiments, the methods comprise: (a) providing genetically modified porcine cells comprising knock-out mutations in each of genes GGTA, CMAH, b4galNT2, SLA-1, and B2M; (b) incubating human cytotoxic cells in the presence and absence of the genetically modified porcine cells of (a); (c) detecting a level of activation or degranulation of the human cytotoxic cells in the presence and absence of the genetically modified porcine cells of (a); wherein, if the level of activation or degranulation of the human cytotoxic cells is increased in the presence of the genetically modified porcine cells as compared to the absence of the genetically modified porcine cells, then the genetically modified porcine cells are xenoreactive. In some embodiments, the cytotoxic cells comprise natural killer (NK) cells. In some embodiments, the cytotoxic cells comprise T cells. In some embodiments, the T cells are CD8⁺ T cells. In some embodiments, the T cells are CD4⁺ T cells. In some embodiments, detecting a level of activation or degranulation of the human cytotoxic cells comprises detecting one or more of: interferon gamma (IFN-g) production, granzyme b expression, surface CD107a, or tumor necrosis factor alpha (TNFa) production. In some embodiments, detecting comprises flow cytometry.

### BRIEF DESCRIPTION OF THE DRAWINGS

Non-limiting embodiments of the presently disclosed compositions, systems, methods, and kits may be further understood by way of example with reference to the accompanying figures.

FIG. 1. Characterization of porcine liver-derived endothelial cells (pLDEC). Porcine cells were analyzed for endothelial cell markers, including cell surface expression of CD31 and intracellular expression of von Willebrand factor (VWF), by flow cytometry. SLA-I and SLA-II expression in pLDEC before and after porcine IFN-7 stimulation were examined by flow cytometric analysis using specific antibodies.

FIG. 2. Phenotype of GM ipLDEC. Flow cytometric analysis of the absence of αGal (A), Sda (C), and SLA-I (D), and reduction of Neu5Gc (B) on ipLDEC (due to uptake of Neu5Gc glycan from Neu5Gc-rich FBS in culture medium).

FIG. 3. Genotype of GM ipLDEC. Each gRNA targeted region was amplified by PCR. Sanger sequencing revealed mutations in GGTA1 (SEQ ID NOs: 32 and 33), CMAH (SEQ ID NOs: 34 and 35), β4galNT2 (SEQ ID NOs: 36 and 37), and B2M (SEQ ID NOs: 38 and 39) genes.

FIG. 4. Comparison of human serum antibody binding to TKO and 5GKO ipLDEC lines. Human sera from 20 patients on the kidney transplant wait-list were incubated with TKO (GGTA1/CMAH/β4galNT2) or 5GKO (GGTA1/CMAH/β4galNT2 SLA-I α chain B2M) ipLDEC. Subsequently, fluorescent conjugated anti-human IgG or IgM was used to evaluate human IgG or IgM binding to the cells, with mean fluorescence intensity (MFI). Student's t-test was used to analyze the differences between TKO and 5GKO ipLDEC lines. Human serum IgG exhibited significant decreased binding to 5GKO ipLDEC compared to TKO ipLDEC (p=0.0026). Human serum IgM binding to 5GKO ipLDEC was also significantly reduced compared to TKO ipLDEC, (p=0.0443).

FIG. 5. Human NK cell degranulation following porcine cell stimulation. Human PBMCs from three donors were treated with human IL-2 (20 ng/mL) for 5 days to expand NK cells, then cocultured with WT, TKO, and 5GKO ipLDEC for 2 hours. Cells were stained with antibodies to CD45, CD3, CD56, and CD107a. Flow cytometry analysis was gated on NK cells (CD3-CD56+) and monitored for the percentage of CD107a positive cells (A) and surface expression of CD107a (MFI) (B). Statistical significance was calculated by one-way ANOVA, Tukey's multiple comparisons test (ns: not significant; ***p<0.001; ****p<0.0001).

### DETAILED DESCRIPTION

### Definitions

To aid in understanding the invention, several terms are defined below.

Unless defined otherwise, all technical and scientific terms used herein have the same meaning as commonly understood by one of skill in the art. Although any methods and materials similar to or equivalent to those described herein can be used in the practice or testing of the claims, the exemplary methods and materials are described herein.

Reference to an element by the indefinite article “a” or “an” does not exclude the possibility that more than one element is present, unless the context clearly requires that there be one and only one element. The indefinite article “a” or “an” thus usually means “at least one.”

The term “about” means within a statistically meaningful range of a value or values such as a stated concentration, length, molecular weight, pH, time frame, temperature, pressure or volume. Such a value or range can be within an order of magnitude, typically within 20%, more typically within 10%, and even more typically within 5% of a given value or range. The allowable variation encompassed by “about” will depend upon the particular system under study.

The terms “comprising,” “having,” “including,” and “containing” are to be construed as open-ended terms (i.e., meaning “including, but not limited to,”) unless otherwise noted.

### Genetically Modified Porcine Cells

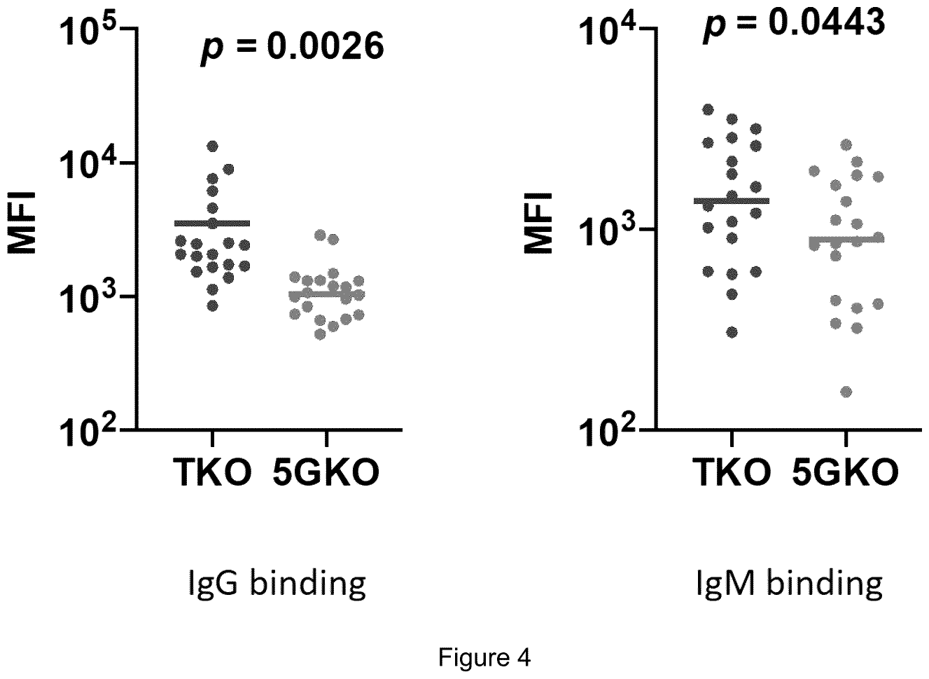

The inventors discovered that genetically modified porcine cells lacking expression of the proteins encoded by the genes GGTA, CMAH, β4galNT2, SLA-1, and B2M have reduced human antibody binding compared to porcine cells lacking expression of the proteins encoded by GGTA, CMAH, and β4galNT2 (FIG. 4).

Accordingly, in a first aspect of the current disclosure, a genetically modified porcine cell is provided. In some embodiments, the genetically modified porcine cells comprise a knock-out mutation in each of the genes GGTA, CMAH, β4galNT2, SLA-1, and B2M.

The gene GGTA encodes the porcine protein N-acetyllactosaminide alpha-1,3-galactosyltransferase which has the amino acid sequence (SEQ ID NO: 1):

| | |
|---|---|
| MNVKGRVVLS MLLVSTVMVV FWEYINSPEG SLFWIYQSKN PEVGSSAQRG WWFPSWFNNG | 60 |
| THSYHEEEDA IGNEKEQRKE DNRGELPLVD WFNPEKRPEV VTITRWKAPV VWEGTYNRAV | 120 |
| LDNYYAKQKI TVGLTVFAVG RYIEHYLEEF LISANTYFMV GHKVIFYIMV DDISRMPLIE | 180 |
| LGPLRSFKVF EIKSEKRWQD ISMMRMKTIG EHILAHIQHE VDFLFCMDVD QVFQNNFGVE | 240 |
| TLGQSVAQLQ AWWYKAHPDE FTYERRKESA AYIPFGQGDF YYHAAIFGGT PTQVLNITQE | 300 |
| CFKGILQDKE NDIEAEWHDE SHLNKYFLLN KPTKILSPEY CWDYHIGMSV DIRIVKIAWQ | 360 |
| KKEYNLVRNN I | 371. |

The gene CMAH encodes the porcine protein cytidine monophosphate-N-acetylneuraminic acid hydroxylase which has the amino acid sequence (SEQ ID NO: 2):

| | |
|---|---|
| MSSIEQTTEI LLCLSPAEAA NLKEGINFVR NKSTGKDYIL FKNKSRLKAC KNMCKHQGGL | 60 |
| FIKDIEDLNG RSVKCTKHNW KLDVSSMKYI NPPGSFCQDE LVVEKDEENG VLLLELNPPN | 120 |
| PWDSEPRSPE DLAFGEVQIT YLTHACMDLK LGDKRMVFDP WLIGPAFARG WWLLHEPPSD | 180 |
| WLERLSRADL IYISHMHSDH LSYPTLKKLA ERRPDVPIYV GNTERPVFWN LNQSGVQLTN | 240 |
| INVVPFGIWQ QVDKNLRFMI LMDGVHPEMD TCIIVEYKGH KILNTVDCTR PNGGRLPMKV | 300 |
| ALMMSDFAGG ASGFPMTFSG GKFTEEWKAQ FIKTERKKLL NYKARLVKDL QPRIYCPFAG | 360 |
| YFVESHPADK YIKETNIKND PNELNNLIKK NSEVVTWTPR PGATLDLGRM LKDPTDSKGI | 420 |
| VEPPEGTKIY KDSWDFGPYL NILNAAIGDE IFRHSSWIKE YFTWAGFKDY NLVVRMIETD | 480 |
| EDFSPLPGGY DYLVDFLDLS FPKERPSREH PYEEIRSRVD VIRHVVKNGL LWDDLYIGFQ | 540 |
| TRLQRDPDIY HHLFWNHFQI KLPLTPPDWK SFLMCSG | 577. |

The gene β4galNT2 encodes the porcine protein beta-1,4-N-acetyl-galactosaminyltransferase 2 which has the sequence (SEQ ID NO: 3):

| | |
|---|---|
| MGSAGFSVGK FHVEVASRGR ECVSGTPECG NRLGSAGFGA LCLELRGADP AWGPFAAHGR | 60 |
| SRRQGSRFLW LLKILVIILV LGIVGFMFGS MFLQAVFSSP KPELPSPAPG VQKLKLLPEE | 120 |
| RLRNLFSYDG IWLFPKNQCK CEANKEQGGY NFQDAYGQSD LPAVKARRQA EFEHFQRREG | 180 |
| LPRPLPLLVQ PNLPFGYPVH GVEVMPLHTV PIPGLQFEGP DAPVYEVTLT ASLGTLNTLA | 240 |
| DVPDSVVQGR GQKQLIISTS DRKLLKFILQ HVTYTSTGYQ HQKVDIVSLE SRSSVAKFPV | 300 |
| TIRHPVIPKL YDPGPERKLR NLVTIATKTF LRPHKLMIML RSIREYYPDL TVIVADDSQK | 360 |
| PLEIKDNHVE YYTMPFGKGW FAGRNLAISQ VTTKYVLWVD DDFLFNEETK IEVLVDVLEK | 420 |
| TELDVVGGSV LGNVFQFKLL LEQSENGACL HKRMGFFQPL DGFPSCVVTS GVVNFFLAHT | 480 |
| ERLQRVGFDP RLQRVAHSEF FIDGLGTLLV GSCPEVIIGH QSRSPVVDSE LAALEKTYNT | 540 |
| YRSNTLTRVQ FKLALHYFKN HLQCAA | 566. |

The gene B2M encodes the porcine protein beta-2-microglobulin which has the amino acid sequence (SEQ ID NO: 4):

| | |
|---|---|
| MAPLVALVLL GLLSLSGLDA VARPPKVQVY SRHPAENGKP NYLNCYVSGF HPPQIEIDLL | 60 |
| KNGEKMNAEQ SDLSFSKDWS FYLLVHTEFT PNAVDQYSCR VKHVTLDKPK IVKWDRDH | 118. |

The gene SLA-1 encodes the porcine major histocompatibility complex type I (also known as swine leukocyte antigen type 1, “SLA type I”), a protein with significant sequence variability. However, an exemplary amino acid sequence for SLA-1 is (SEQ ID NO: 5):

| | |
|---|---|
| MGPGALFLLL SGTLALTGTQ AGPHSLSYFY TAVSRPDRGD SRFIAVGYVD DTQFVRFDNY | 60 |
| APNPRMEPRV PWIQQEGQEY WDRETRNVKE TAQTYGVGLN TLRGYYNQSE AGSHTLQSMY | 120 |
| GCYLGPDGLL LHGYRQDAYD GADYIALNED LRSWTAADMA AQITKRKWEA ADEAERRRSY | 180 |
| LQGLCVESLR RYLEMGKDTL QRAEPPKTHV TRHPSSDLGV TLRCWALGFY PKEISLTWQR | 240 |
| EGQDQSQDME LVETRPSGDG TFQKWAALVV PPGEEQSYTC HVQHEGLQEP LTLRWDPAQP | 300 |
| PVPIVGIIVG LVLVLVAGAM VAGVVIWRKT RSGEKGGSYT QAAGSDSDQG SDVSLTKDPR | 360 |
| V | 361. |

As used herein, “knock-out mutation” or “knocked-out” refers to a mutation in a gene in the genome of a cell or organism that causes reduced or absence of expression of the protein encoded by the gene. In other words, knock-out mutations are mutations in a protein coding gene that eliminates or essentially eliminates expression or function of the encoded protein. In some embodiments, the mutation may be made in a region of the gene that is not protein coding, but rather, that is required for normal transcription or translation of the product of the gene, e.g., splice acceptor sites, splice donor sites, or promoters that are operably linked to the gene. In general, knock-out mutations that result in production of no protein are contemplated by the instant disclosure, e.g., mutations in GGTA, CMAH, β4galNT2, SLA-1, and B2M that result in no N-acetyllactosaminide alpha-1,3-galactosyltransferase, cytidine monophosphate-N-acetylneuraminic acid hydroxylase, beta-1,4-N-acetyl-galactosaminyltransferase 2, swine lymphocyte antigen 1, and beta-2-microglobulin, respectively.

In some embodiments, knock-out of the gene CMAH may refer to a mutation that produces a non-functional, or essentially non-functional, gene product. CMAH encodes the protein cytidine monophosphate-N-acetylneuraminic acid hydroxylase which hydroxylates N-acetylneuraminic acid to produce N-glycolylneuraminic acid. CMAH expression has been lost in humans and exists as a pseudogene with no detectable functional gene product. As a result, normal human tissue does not comprise N-glycolylneuraminic acid. Thus, by knocking out CMAH, the inventors remove the enzymatic function of generating N-glycolylneuraminic acid in porcine cells or tissue. Accordingly, in the case of CMAH, a knock-out may comprise a mutation that disrupts the function of the gene product but does not prevent expression of a gene product.

Gene “knock-outs” may be generated by a variety of methods known in the art; however, an exemplary method, and the method selected by the inventors to generate the disclosed genetically modified porcine cells, is the use of CRISPR/Cas9 nucleic acid-targeted nuclease technology. The use of CRISPR/Cas9 to generate gene knock-outs is known in the art. Exemplary guide RNA targeting sites are provided that were used to target each of GGTA, CMAH, β4galNT2, SLA-1, and B2M in Table 1 (SEQ ID NOs: 6-11). Such sequences can be inserted into existing vectors that comprise the rest of the guide RNA scaffold that have been developed for expression in mammalian cells. The cells must also comprise Cas9 nuclease, whereupon the gRNA and Cas9 in complex will bind to the targeted DNA locus and create a double-stranded break (DSB). The DSB may be repaired by error-prone non-homologous end joining, thus, introducing insertions/deletions (indels) that may shift the reading frame and/or introduce a stop codon. Methods used to by the inventors to generate the knock-out alleles of the instant disclosure are discussed further and in more detail in Example 1. In addition, further teaching is provided below in the sections entitled “CRISPR/Cas” and “Cas9” discussing potential variations of nucleic-acid guided nuclease systems and additional direction for their use.

In the methods and compositions disclosed herein, the “knock-out” mutations in the disclosed cells, tissues, organoids, or organisms are effectively homozygous, in that both copies of the gene of interest are non-functional or not expressed, or are essentially non-functional or essentially not expressed. “Essentially” as used in this context means that any expression or function detected is so low as to be ineffective or irrelevant.

The inventors have previously demonstrated that porcine liver-derived endothelial cells, which express both CD31 and von Willebrand factor (VFW) can be successfully used in the generation of genetically modified pigs by the somatic cell nuclear transfer (SCNT) technique. See, for example, Adams, A. B. et al. Xenoantigen Deletion and Chemical Immunosuppression Can Prolong Renal Xenograft Survival. Ann Surg 268, 564-573, doi:10.1097/SLA.0000000000002977 (2018), Li, P. et al. Efficient generation of genetically distinct pigs in a single pregnancy using multiplexed single-guide RNA and carbohydrate selection. Xenotransplantation 22, 20-31, doi:10.1111/xen.12131 (2015), Estrada, J. L. et al. Evaluation of human and non-human primate antibody binding to pig cells lacking GGTA1/CMAH/beta4GalNT2 genes. Xenotransplantation 22, 194-202, doi:10.1111/xen.12161 (2015), and Paris, L. L. et al. Reduced human platelet uptake by pig livers deficient in the asialoglycoprotein receptor 1 protein. Xenotransplantation 22, 203-210, doi:10.1111/xen.12164 (2015). Therefore, in some embodiments, the instantly disclosed cell may be a porcine liver-derived endothelial cell (pLDEC), which, in some embodiments, expresses CD31 and von Willebrand factor.

Though pLDECs are exemplified in the instant disclosure, additional or alternative cell types are envisioned by the inventors including, but not limited to, fibroblasts, hematopoietic stem progenitor cells, induced pluripotent stem cells, embryonic stem cells, cardiomyocytes, hepatocytes, hepatic stellate cells, cholangiocytes, Kuppfer cells, renal cells, endothelial cells, pneumocytes, etc.

In some embodiments, the porcine cells of the instant disclosure exhibit reduced immunoreactivity to a human serum sample compared to a genetically modified porcine cell comprising knock-out mutations only in genes GGTA, CMAH, and β4galNT2. As used herein, “immunoreactivity” refers to the property of being activated or able to bind and capable of initiating an immune reaction in the presence of target. In some embodiments, cells or tissue are immunoreactive if they activate cytotoxic cells, e.g., human cytotoxic cells, while, in other embodiments, cells or tissue are immunoreactive if they are bound by antibodies, human antibodies.

In some embodiments, the cell of the instant disclosure is immortalized such that it can be passaged for 10 times, 20 times, 30 times, 40 times, 50 times, 60 times, 70 times, 80 times, 90 times, or 100 times or more before losing critical characteristics of the cell, e.g., CD31 and VWF expression. A variety of methods to immortalize a porcine cell are known in the art and include, but are not limited to expression of: one or more immortalization factor selected from SV40 T antigen, human telomerase (hTERT), simian virus 40 large T antigen, papillomavirus E6, papillomavirus E7, adenovirus E1A, Epstein-Barr virus, and human T-cell leukemia virus in the cell.

In another aspect of the current disclosure, animals comprising the disclosed genetically modified cells comprising a knock-out mutation in each of the genes GGTA, CMAH, β4galNT2, SLA-1, and B2M. In some embodiments, the animals comprising the disclosed cells include, but are not limited to, a human being, a cow, a horse, a sheep, a pig, a monkey, an ape, or a rodent.

One advantageous use for the instantly disclosed cells and methods is to provide data with more translational relevance for preclinical trials and accelerate the application of xenotransplantation (XTx) in humans.

### Organoids

In another aspect of the current disclosure, organoids are provided. In some embodiments, the organoids comprise a genetically modified porcine cell comprising a knock-out mutation in each of genes GGTA, CMAH, β4galNT2, SLA-1, and B2M. As used herein, “organoid” is a 3-dimensional multicellular in vitro tissue construct that mimics characteristics of its corresponding in vivo organ, such that it can be used to study aspects of that organ in vitro. Organoids typically self-assemble and may be differentiated in vitro from stem cells. In contrast, spheroids are spherical cellular units that are generally cultured as free-floating aggregates and are arguably of low complexity in mirroring organ organization. However, both organoids as well as spheroids comprising a genetically modified porcine cell comprising a knock-out mutation in each of genes GGTA, CMAH, β4galNT2, SLA-1, and B2M are contemplated herein. In some embodiments, porcine endothelial cells and fibroblasts isolated from the same pig are used to generate spheroids at ratio of 1:3, respectively. For each spheroid, the 40,000-cell mixture is plated in a 96-well U-bottom low affinity binding plate and cultured for 2 days. In some embodiments, the organoids are liver organoids and comprise hepatocytes, hepatic stellate cells, and liver sinusoidal endothelial cells at a ratio of 2.5:1:1, respectively. In some embodiments, the liver organoids comprise hepatocytes, hepatic stellate cells, liver sinusoidal endothelial cells, optionally at a ratio of 2.5:1:1, respectively, and Kupffer cells and cholangiocytes. In some embodiments, the spheroids are porcine lung spheroids and comprise CD31⁺ pulmonary vascular endothelial cells, pulmonary fibroblasts, and pulmonary pneumocytes Type II at a ratio of 1:1:0.5, respectively. In some embodiments, the spheroids are porcine pancreatic spheroids and comprise porcine alpha cells, beta cells, fibroblasts, and endothelial cells. In some embodiments, the spheroids are porcine kidney spheroids and comprise porcine fibroblasts, endothelial cells, and proximal tubule epithelial cells. In some embodiments, the spheroids are porcine corneal spheroids and comprise porcine corneal endothelial cells, stromal cells, and corneal epithelial cells.

### Porcine Tissue

In another aspect of the current disclosure, porcine tissues are provided. In some embodiments, the porcine tissues comprise a genetically modified porcine cell comprising a knock-out mutation in each of genes GGTA, CMAH, β4galNT2, SLA-1, and B2M. In some embodiments, the tissues may include, but are not limited to, heart tissue, e.g., myocardium or heart valves, liver tissue, muscle tissue, retinal tissue, corneal tissue, kidney tissue, skin tissue, pancreatic tissue, connective tissue, e.g., ligaments, tendons, or cartilage, etc. In some embodiments, the tissues are grown in vitro from genetically modified porcine cells. In some embodiments, the disclosed tissues are derived from genetically modified pigs. In some embodiments, the tissue is comprised entirely of, or derived from the disclosed genetically modified porcine cells. In some embodiments, the tissues comprise the genetically modified cells of the instant disclosure and also include additional porcine cells, or non-porcine cells. See, for example, Kim et al. “The Porcine Aortic Tissue Culture System in vitro for Stem Cell Research” Int J Stem Cells. 2011 November; 4(2): 116-122 and Ou et al. “Physiological Biomimetic Culture System for Pig and Human Heart Slices” Circ Res. 2019 Aug. 30; 125(6): 628-642, each of which are incorporated by reference herein in their entireties.

In another aspect of the current disclosure, in vitro cultured porcine tissue suitable for transplantation is provided. In some embodiments, the in vitro cultured porcine tissue suitable for transplantation comprises genetically modified porcine cells comprising a knock-out mutation in each of genes GGTA, CMAH, β4galNT2, SLA-1, and B2M.

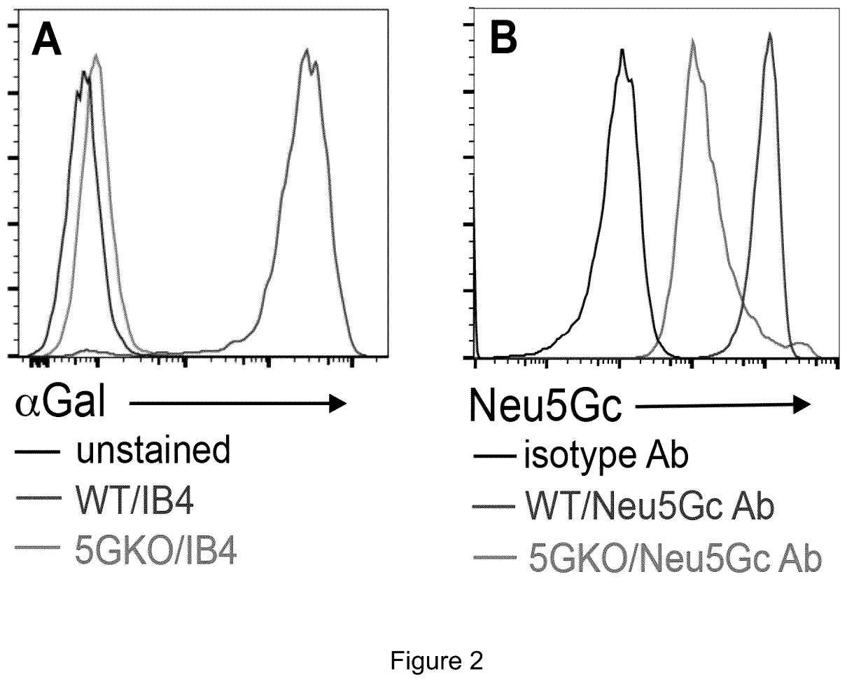

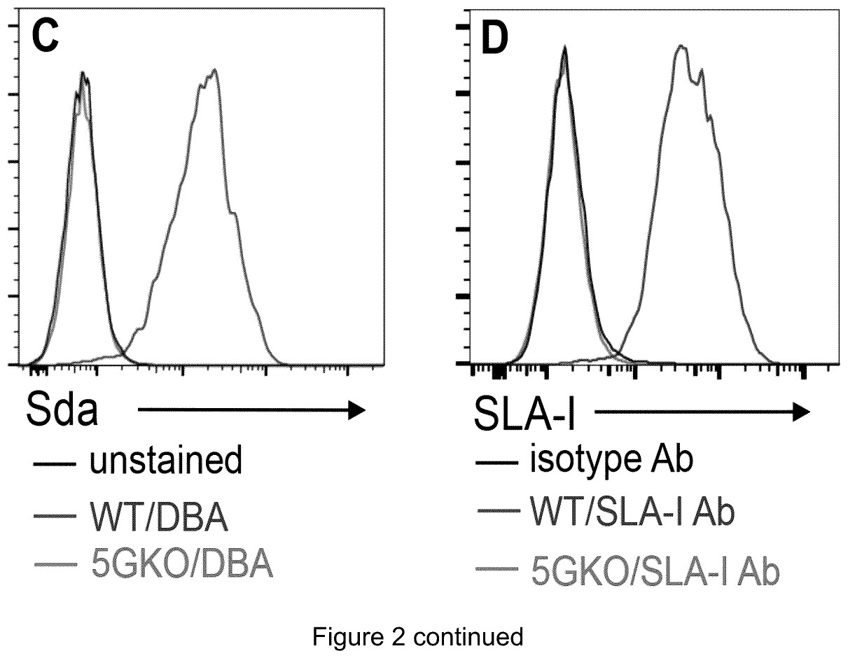

As used herein, “suitable for transplantation” refers to the property of being medically acceptable for transplantation into a host. In some embodiments, transplantation is xenotransplantation into a host, e.g., a human being, an ape, a monkey, a rodent, or a ruminant mammal. In some embodiments, tissues that are suitable for transplantation are essentially free of cell culture contaminants, e.g., bovine or horse serum, and free of pathogenic or free of potentially pathogenic microorganisms, e.g., commensal microorganisms or opportunistic microorganisms. It is known in the art that cells or tissue cultured in serum comprising N-glycolylneuraminic acid, e.g., bovine serum, horse serum, etc., will accumulate N-glycolylneuraminic acid even if said cells or tissue have knocked-out CMAH. See, e.g., FIG. 2B. Thus, in some embodiments, especially where the transplant recipient is a human, cells or tissue suitable for transplantation are essentially free of N-glycolylneuraminic acid.

As used herein, “essentially free of” refers levels of a specific target, e.g., N-glycolylneuraminic acid, that are below the level of detection with a suitable assay (e.g., an ELISA assay).

### Genetically Modified Pigs

In another aspect of the current disclosure, genetically modified pigs are provided. In some embodiments, the genetically modified pigs comprise a genetically modified porcine cell comprising a knock-out mutation in each of genes GGTA, CMAH, β4galNT2, SLA-1, and B2M. The inventors have previously demonstrated the successful generation of live born genetically modified pigs created by genetically modifying pLDECs with CRISPR/Cas9 technology in conjunction with somatic cell nuclear transfer (SCNT) techniques. See, for example, Ping, L. et al., 2015, supra; Lutz et al. “Double knockout pigs deficient in N-glycolylneuraminic acid and galactose α-1,3-galactose reduce the humoral barrier to xenotransplantation” Xenotransplantation. January-February 2013; 20(1):27-35; Li et al. “Biallelic knockout of the α-1,3 galactosyltransferase gene in porcine liver-derived cells using zinc finger nucleases” J Surg Res. 2013 May 1; 181 (1):e39-45; Paris et al. “Reduced human platelet uptake by pig livers deficient in the asialoglycoprotein receptor 1 protein” Xenotransplantation. May-June 2015; 22(3):203-10; Estrada et al. “Evaluation of human and non-human primate antibody binding to pig cells lacking GGTA1/CMAH/β4GalNT2 genes” Xenotransplantation. May-June 2015; 22(3):194-202; Martens et al. “Humoral Reactivity of Renal Transplant-Waitlisted Patients to Cells From GGTA1/CMAH/B4GalNT2, and SLA Class I Knockout Pigs” Transplantation. 2017 April; 101(4):e86-e92; Adams et al. “Xenoantigen Deletion and Chemical Immunosuppression Can Prolong Renal Xenograft Survival” Ann Surg. 2018 October; 268(4):564-573; and Martens et al. “HLA Class I-sensitized Renal Transplant Patients Have Antibody Binding to SLA Class I Epitopes” Transplantation. 2019 August; 103(8):1620-1629; each of which are incorporated by reference herein in their entireties.

In addition to the genetically modified pigs disclosed herein, the inventors also envision the use of tissues from said pigs as transplant material for xenotransplantation to a host, e.g., a human being. In some embodiments, the tissues may include, but are not limited to, heart tissue, e.g., myocardium or heart valves, liver tissue, muscle tissue, retinal tissue, corneal tissue, kidney tissue, skin tissue, pancreatic tissue, connective tissue, e.g., ligaments, tendons, or cartilage, etc. In some embodiments, whole organs are transplanted into a xenogeneic host, e.g., a human being or an ape or monkey. In some embodiments, the organs include, but are not limited to, liver, heart, kidney, lung, etc.

### CRISPR/Cas

CRISPR/Cas system proteins include proteins from CRISPR Type I systems, CRISPR Type II systems, and CRISPR Type III systems. CRISPR/Cas system proteins can be from any bacterial or archaeal species. In some embodiments, the CRISPR/Cas system proteins are from, or are derived from CRISPR/Cas system proteins from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, or Corynebacter diphtheria.

The nucleic acid-guided nuclease proteins can be naturally occurring or engineered versions. Naturally occurring CRISPR/Cas system proteins include Cas9, Cpf1, Cas3, Cas8a-c, Cas10, Cse1, Csy1, Csn2, Cas4, Csm2, and Cm5. In an exemplary embodiment, the CRISPR/Cas system protein comprises Cas9.

A “CRISPR/Cas system protein-gRNA complex” refers to a complex comprising a CRISPR/Cas system protein and a guide RNA. The guide RNA may be composed of two molecules, i.e., one RNA (“crRNA”) which hybridizes to a target and provides sequence specificity, and one RNA, the “tracrRNA”, which is capable of hybridizing to the crRNA. Alternatively, the guide RNA may be a single molecule (i.e., a gRNA) that contains crRNA and tracrRNA sequences. A CRISPR/Cas system protein may be at least 60% identical (e.g., at least 70%, at least 80%, or 90% identical, at least 95% identical or at least 98% identical or at least 99% identical) to a wild type CRISPR/Cas system protein. The CRISPR/Cas system protein may have all the functions of a wild type CRISPR/Cas system protein, or only one or some of the functions, including binding activity, nuclease activity, and nicking activity.

The term “CRISPR/Cas system protein-associated guide RNA” refers to a guide RNA as described above (comprising a crRNA molecule and a tracrRNA molecule or comprising a single RNA molecule that includes both crRNA and tracrRNA sequences). The CRISPR/Cas system protein-associated guide RNA may exist as isolated RNA, or as part of a CRISPR/Cas system protein-gRNA complex.

### Cas9

The CRISPR/Cas system protein used in the methods may comprise Cas9. The Cas9 of the present invention can be isolated, recombinantly produced, or synthetic. Cas9 proteins that can be used in the embodiments herein can be found in http://www.nature.com/nature/journal/v520/n7546/full/nature14299.html.

In some embodiments, the Cas9 is a Type II CRISPR system derived from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, or Corynebacter diphtheria.

In some embodiments, the Cas9 is a Type II CRISPR system derived from S. pyogenes and the PAM sequence is NGG located on the immediate 3′ end of the target specific guide sequence. The PAM sequences of Type II CRISPR systems from exemplary bacterial species can also include: Streptococcus pyogenes (NGG), Staph aureus (NNGRRT), Neisseria meningitidis (NNNNGA TT), Streptococcus thermophilus (NNAGAA) and Treponema denticola (NAAAAC) which are all usable without deviating from the present invention. ((http://www.nature.com/nature/journal/v520/n7546/full/nature14299.html)).

In one exemplary embodiment, Cas9 sequence can be obtained, for example, from the pX330 plasmid (available from Addgene), re-amplified by PCR then cloned into pET30 (from EMD biosciences) to express in bacteria and purify the recombinant 6His tagged protein.

The “Cas9-gRNA complex” refers to a complex comprising a Cas9 protein and a guide RNA and is one example of a CRISPR/Cas system protein-gRNA complex. A Cas9 protein may be at least 60% identical (e.g., at least 70%, at least 80%, or 90% identical, at least 95% identical or at least 98% identical or at least 99% identical) to a wild type Cas9 protein, e.g., to the Streptococcus pyogenes Cas9 protein. The Cas9 protein may have all the functions of a wild type Cas9 protein, or only one or some of the functions.

The term “Cas9-associated guide RNA” refers to a guide RNA as described above (comprising a crRNA molecule and a tracrRNA molecule or comprising an RNA molecule that includes both crRNA and tracrRNA sequences). The Cas9-associated guide RNA may exist as isolated RNA, or as part of a Cas9-gRNA complex.

### Methods of Testing Genetically Modified Porcine Cells for Xenoreactivity

In another aspect of the current disclosure, methods of testing genetically modified porcine cells for xenoreactivity are provided. As used herein, “xenoreactive” or “xenoreactivity”, similarly to immunoreactive or immunoreactivity, refers to the property of being activated or able to bind and capable of initiating an immune reaction in the presence of target, e.g., genetically modified cells, tissues, or the like, in the case where the target is xenogeneic, i.e., is from another species. Thus, xenoreactivity may be defined as immunoreactivity relative to another species. In some embodiments, xenoreactivity is determined relative to human cytotoxic cells and antibodies. Thus, in some embodiments, cells or tissue are xenoreactive if they activate cytotoxic cells, e.g., human cytotoxic cells, while, in other embodiments, cells or tissue are xenoreactive if they are bound by antibodies, human antibodies. Accordingly, xenoreactivity is relative to the intended host organism of the xenogeneic donor cells or tissue. Thus, if the contemplated host of xenoreactive cell or tissue is a human, then the xenoreactivity of the intended donor cells or tissues should be determined using human cytotoxic cells or human antibodies.

In some embodiments, the methods comprise (a) providing genetically modified porcine cells comprising knock-out mutations in each of genes GGTA, CMAH, β4galNT2, SLA-1, and B2M; (b) incubating human cytotoxic cells in the presence and absence of the genetically modified porcine cells of (a); (c) detecting a level of activation or degranulation of the human cytotoxic cells in the presence and absence of the genetically modified porcine cells of (a); wherein, if the level of activation or degranulation of the human cytotoxic cells is increased in the presence of the genetically modified porcine cells as compared to the absence of the genetically modified porcine cells, then the genetically modified porcine cells are xenoreactive.

In some embodiments, the genetically modified porcine cells comprise at least one additional mutation, e.g., knock-out or transgenic expression. In such embodiments, the methods described herein may be performed to test whether the at least one additional mutation reduces xenoreactivity of the genetically modified porcine cells.

In some embodiments, detecting a level of activation or degranulation of the human cytotoxic cells comprises detecting one or more of: interferon gamma (IFN-γ) production, granzyme B expression, surface CD107a, or tumor necrosis factor alpha (TNFα) production, which are well known in the art and my utilize, for example, flow cytometry to detect the production of IFN-γ, TNFα intracellularly, surface expression of CD107a secondary to degranulation, and/or the level of expression of granzyme B which may fluctuate in response to activation state or differentiation state of certain cytotoxic cells.

In some embodiments, the cytotoxic cells are natural killer cells. In humans, NK cells are typically defined as CD3⁻CD56⁺ cells that are also CD7⁺CD127⁻NKp46⁺T-bet⁺Eomes⁺. NK cells lack a specific antigen receptor and are instead induced by a variety of receptors present on their surface, in addition to sensing of the cytokine milieu.

In some embodiments, the cytotoxic cells are T cells. T cells are typically characterized by the markers CD3⁺, and either CD4⁺ or CD8⁺. T cells require ligation of the T cell receptor (TCR) to Major histocompatibility complex class I (MHC class I) or class II (MHC class II), for CD8⁺ and CD4⁺ T cells, respectively. Thus, in contrast to NK cells, for successful testing of T cells, the xenogeneic donor cells must also express the appropriate MHC, which in humans is referred to as human lymphocyte antigen (HLA).

In some embodiments, the methods of testing genetically modified porcine cells for xenoreactivity comprise (a) providing genetically modified porcine cells comprising knock-out mutations in each of genes GGTA, CMAH, β4galNT2, SLA-1, and B2M; (b) incubating the genetically modified porcine cells in the presence and absence of human antibodies; (c) detecting a level of binding of human antibodies in the presence and absence of the genetically modified porcine cells of (a); wherein, if the level of binding of human antibodies is increased in the presence of the genetically modified porcine cells as compared to the absence of the genetically modified porcine cells, then the genetically modified porcine cells are xenoreactive. In some embodiments, detecting comprises incubating the genetically modified porcine cells with labelled anti-human antibodies. As used herein, “labelled” refers to the property of comprising a detectable label. Exemplary detectable labels include, but are not limited to, fluorescent labels, chemiluminescent labels, radioactive labels, colorimetric labels, etc.

Recitation of ranges of values herein are merely intended to serve as a shorthand method of referring individually to each separate value falling within the range, and includes the endpoint boundaries defining the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### EXAMPLES

The following examples are illustrative and should not be interpreted to limit the scope of the claimed subject matter.

### Example 1: Porcine Cells Having Reduced Antigenicity

Pig-to-NHPs pre-clinical XTx trials are essential for the clinical application of XTx. However, this model has some limitations. Different GM pigs have to be generated to prevent immune rejection in NHPs and humans, due to species disparity between humans and NHPs.

Baboons and rhesus macaques have been used as recipients in different laboratories, which make the results hard to compare. Therefore, evaluation of the human immune responses to porcine cells with various genetic modifications may provide a straightforward approach to identify the benefit of genetic modifications.

In the present study, four xenoantigens (αGal, Neu5Gc, Sda, and SLA-I) were depleted in a porcine endothelial cell line using CRISPR/Cas9. This engineered porcine cell line exhibited minimum/limited reactivity to human antibodies but triggered human NK cell activation. The human anti-pig humoral and cellular responses are significant impediments to success in XTx.

This engineered porcine cell line can be used for further genetic modification to improve xenograft compatibility and test human immune responses in vitro. We chose an ipLDEC line for the following reasons; (i) porcine endothelial cell activation and damage are the first episode of xeno-rejection²¹, (ii) porcine endothelial cells not only express all three carbohydrate xenoantigens⁹, but also express SLA-II and elevated SLA-I molecules upon proinflammatory cytokine stimulation, (iii) both SLA-I and SLA-II molecules have been identified as xenoantigensi^{6,22}. Porcine endothelial cells are vulnerable to antibody-mediated destruction, compared to porcine fibroblasts. Porcine endothelial cells can also be activated by human proinflammatory cytokines, IL-6, IL-17, IL-1b, and TNF-α23. In addition, porcine endothelial cells express activating ligands for human lymphocyte-activating receptor NKG2D to trigger NK cell- and T cell-mediated cytotoxicity.

This pLDEC line¹⁷ has been previously used to generate multiple GM pigs in our laboratory^{4,8,9,11,18} Human antibody reactivity to PBMCs from GGTA1 KO, GGTA1/CMAH DKO, GGTA1/CMAH/β4galNT2 TKO pigs has been comprehensively examined^{19,22}. Our results indicated that human antibody reactivity to TKO ipLDEC is consistent and comparable to human antibody reactivity to PBMCs from TKO pig. A previous study indicated that 69% of 820 renal transplant waitlist patients had antibodies against PBMC from TKO pigs, some of antibodies were HLA class I antibodies cross-reacting with SLA-I molecules¹⁶. Therefore, removal of SLA-I in a TKO pig is necessary for highly sensitized patients. They have little chance to obtain a matched renal allograft but may benefit from a pig organ that does not express SLA-I, αGal, Neu5Gc, and Sda xenoantigens. SLA-I-deficient pigs have been generated by several groups^{10,25,26}.

Fischer et al reported that porcine fibroblasts derived from pigs deficient in SLA-I, αGal, Neu5Gc, and Sda xenoantigens, exhibited further decreased human antibody reactivity¹⁰, which is consistent with the results of our study. 5GKO ipLDEC line has significantly decreased human IgG and IgM reactivity, compared to the TKO ipLDEC line. TKO and 5GKO ipLDEC are valuable reagents and ready to use in a crossmatch assay, avoiding the generation of TKO and 5GKO pigs and isolation of porcine cells to examine antibodies in patients.

SLA-I-deficient porcine cells were generated by the disruption of SLA-I α chains and B2M genes. SLA-I genes (α chains) are highly polymorphic with 227 alleles designated to date (https://www.ebi.ac.uk/ipd/mhc/group/SLA/). The SLA-I gene cluster contains three constitutively expressed classical genes, SLA-1, SLA-2, and SLA-3. Cas9 and two gRNAs targeting the common regions of multiple alleles in SLA-1, SLA-2, and SLA-3 completely abolished SLA-I expression. B2M was subsequently disrupted and the mutation was confirmed by Sanger sequencing. Since we subsequently plan to express human HLA class I molecules in the 5GKO ipLDEC line, removal of both SLA-I α chains and B2M is required. Complete elimination of two subunits of SLA-I will prevent the formation of SLA-I or HLA-class I hybrids, because SLA-I α chain pairing with human B2M to rescue SLA-I expression has been found (unpublished data).

Human NK cell is one of the components of the innate immune system, and is involved in xenograft rejection. NK cell infiltration was found in pig organs perfused with human blood^{27,28} and in pig-to-NHP xenografts^{29,30}. A dual role of NK cells in allograft transplantation by both graft rejection and tolerance has also been revealed³¹. In an allogeneic setting, self-MHC class I is a signal for NK cell tolerance. Whether SLA-I can serve as an inhibitory ligand, as its counterpart HLA class I, to human NK cell activation is uncertain. Sullivan et al reported that SLA-I is unable to efficiently transmit inhibitory signals to human NK cells because amino acid residues critical for the binding to human NK cell inhibitory receptors are altered in SLA-I, when compared to HLA class I.³² Another study showed that the induction of SLA-I expression on porcine endothelium by tumor necrosis factor-alpha (TNF-α) reduced lysis by human NK cells. We compared the human NK cell response to SLA-I-deficient porcine cells and porcine cells with functional SLA-I. Our results showed that human NK cells became activated by porcine cell stimulation, and the absence of SLA-I on porcine cells failed to trigger stronger NK cell activation. A similar observation was reported in a recent study using porcine cells with decreased SLA-I (SLA-Ilow) expression.²⁵ Taken together, these results demonstrated that SLA-I is not an inhibitory ligand for human NK cells.

Human NK cell activation was monitored by flow cytometric analysis of CD107a surface expression. CD107a or lysosome-associated membrane protein-1 (LAMP-1) is a marker of immune cell activation and cytotoxic degranulation. A previous study indicated that CD107a surface expression has a direct correlation with NK cell cytotoxic activity and cytokine secretion, which is a reliable marker for evaluation of activation of immune cell cytotoxic responses against stimuli such as xenogeneic cells.

If antibody-mediated immune rejection can be controlled, prevention of the cellular immune rejection becomes the main focus in XTx research. In pig-to-NHP preclinical XTx trials, various immunosuppressants have been used to prevent the cellular immune response, which are often either not clinically applicable or not practical due to deleterious side-effects.

Development of a novel way to induce NK cell immune tolerance and improve human-pig compatibility is needed to speed XTx application. Manipulation of NK ligands on porcine cells, including removal of activating ligands and introducing inhibitory ligands, may protect xenografts from immune cell-mediated cytotoxicity. TKO and 5GKO ipLDEC lines are valuable for further genetic engineering to mitigate human cellular immune responses, which will ultimately contribute to long-term survival of pig xenografts in humans.

### ABBREVIATIONS

B2M: β2-microglobulin
B4galNT2: Beta-1,4-N-acetyl-galactosaminyltransferase 2
CMAH: Cytidine monophospho-N-acetylneuraminic acid hydroxylase
GGTA1: alpha-1,3-galactosyltransferase
GM: genetic modification
HLA: human leukocyte antigen
MHC: major histocompatibility complex
Neu5Gc: N-glycolylneuraminic acid
NK: natural killer
PBMC: peripheral blood mononuclear cells
SLA: swine leukocyte antigen

### Results

### Characterization and Immortalization of Porcine Liver-Derived Endothelial Cells.

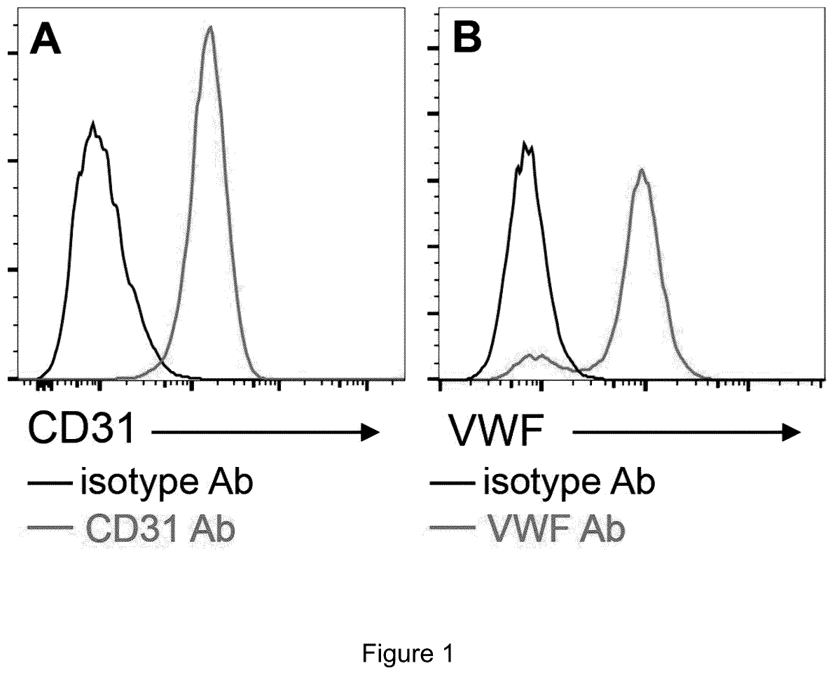

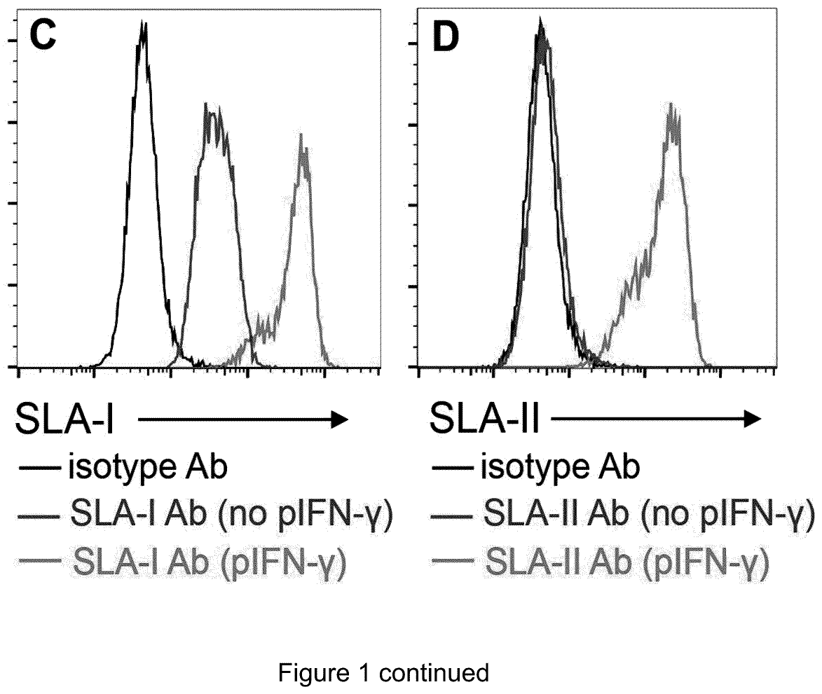

A primary porcine liver-derived cell exhibited endothelial cell-like morphology¹⁷, which had been previously used for multiple-gene modifications and various GM pig production, including GGTA1/β4galNT2 DKO⁴, GGTA1/CMAH/iGb3S TKO⁸, GGTA1/CMAH/β4galNT2 TKO⁹, and GGTA1/ASGR1 DKO¹⁸. Flow cytometric analysis showed that this cell line expressed endothelial markers CD31 (FIG. 1A) and von Willebrand factor (VWF) (FIG. 1). Upon porcine IFN-7 stimulation, SLA-I expression was dramatically upregulated (FIG. 1.C) and SLA-II was detected on the cell surface (FIG. 1.D). Together, expression of CD31 and VWF endothelial markers along with de novo expression of MHC class II molecules and increased surface expression of MHC class I molecules responding to porcine IFN-7 stimulation, confirmed porcine liver-derived endothelial cells (pLDEC). In order to make various genetic modifications and to test the human immune response to these genetic modifications, pLDEC line was immortalized by lentivirus expressing the SV40 T antigen and hTERT to obtain an immortalized pLDEC line (ipLDEC)

Establishing a Five Gene-Knockout (5GKO: GGTA1/CMAH/β4galNT2 SLA-Iα Chain B2M) ipLDEC Line.

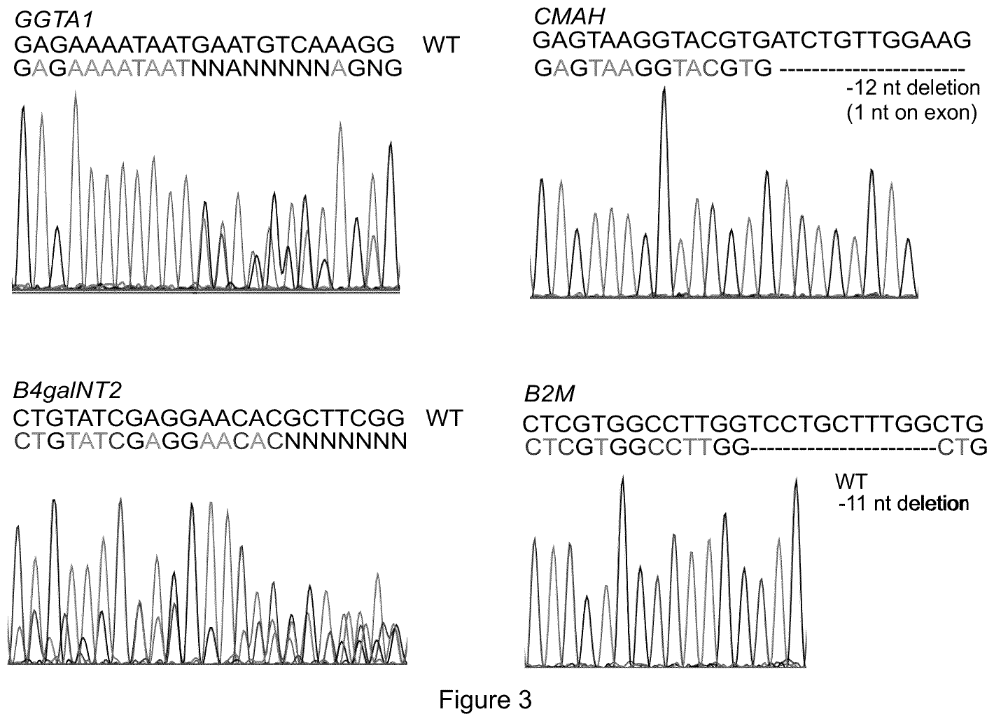

ipLDEC was co-transfected with CRISPR/Cas9 plasmids with guide-RNA (gRNA) targeted to GGTA1, CMAH, and β4galNT2 genes (Table 1), respectively. After sequential phenotypic selection, an ipLDEC line null for αGal, Neu5Gc, and Sda (TKO) was obtained (FIG. 2.A-C). The gRNA targeted region of each gene was amplified by PCR using specific primers (Table 2). The mutations of GGTA1, CMAH, and β4galNT2 genes were confirmed by Sanger sequencing (FIG. 3). Due to uptake and metabolic incorporation of the Neu5Gc glycan from Neu5Gc-rich FBS in culture media, some Neu5Gc was detected in TKO ipLDEC, but it was significantly less than expression of Neu5Gc in WT ipLDEC (MFI: 28509 vs 105646) (FIG. 2B). This is a very common phenomenon when culturing TKO cells in medium with FBS.

SLA-I molecules consist of polymorphic α chains and conserved β2-microglobulin (B2M). To completely remove SLA-I in porcine cells, we designed gRNA targeting both α chains and B2M genes. TKO ipLDEC line was transfected with plasmids containing two selected gRNAs targeting the common sequences in multiple alleles of SLA-I α chains. Disruption of SLA-I α chains led to the loss of SLA-I expression, so the mutant cells were selected by flow cytometry cell sorting (FIG. 2D).

This four-gene-knockout cell line (GGTA1/CMAH/β4galNT2 SLA-I α chain) was transfected with the plasmid bearing gRNA targeting the B2M gene. B2M mutants were screened by PCR and Sanger sequencing, and a clone with 11 nucleotide-deletion was selected (FIG. 3). Thus, an ipLDEC line with 5-gene-knockout (5GKO; GGTA1/CMAH/β4galNT2 SLA-I α chain/B2M) was successfully established.

Reduced Human Antibody Reactivity to 5GKO ipLDEC.

Previous studies indicated that some human HLA class I antibodies cross-react with swine SLA-I molecules^{16,19,20}. Elimination of SLA-I may diminish HLA class I antibody binding to prevent antibody-mediated rejection. We compared human antibody reactivity to TKO and 5GKO ipLDEC lines. Human serum samples were from 20 patients on the kidney transplant wait-list with panel-reactive antibody (PRA) in the range of low (<10%, n=2) to high (>90%, n=18). Human IgG binding to 5GKO ipLDEC was significantly reduced compared to human IgG binding to TKO ipLDEC (p=0.0026). Human IgM reactivity to 5GKO ipLDEC compared to TKO ipLDEC was also statistically decreased (p=0.0443) (FIG. 4).

Activation of Human Natural Killer (NK) Cells by 5GKO ipLDEC.

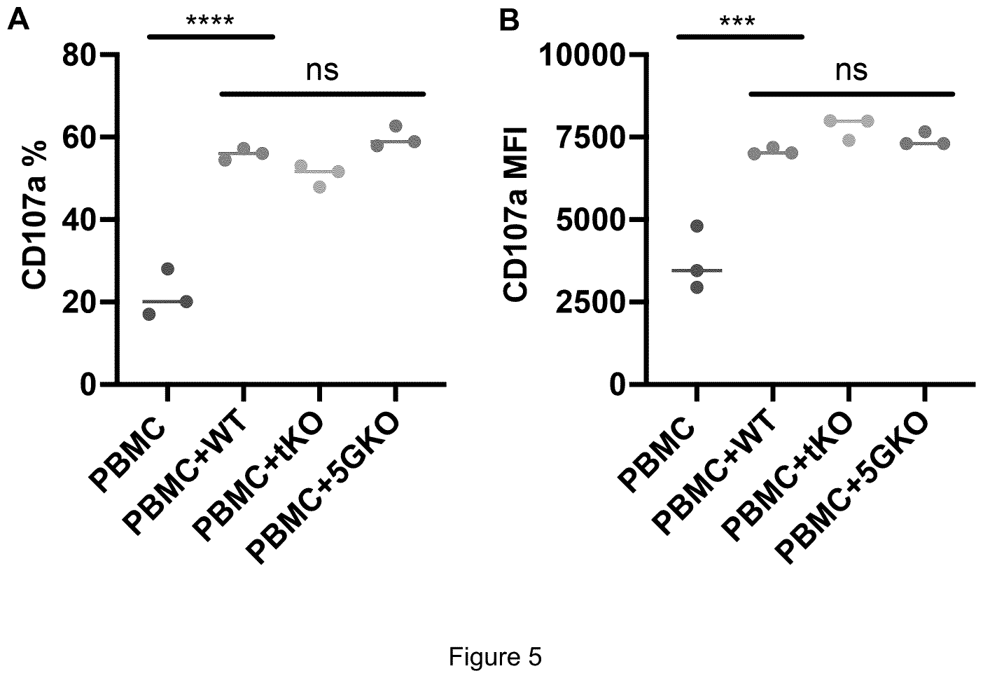

NK cells can directly recognize and lyse target cells through their receptors interacting with the ligands on target cells. Human PBMCs were isolated from the ‘buffycoats’ of three individuals and pre-treated with human TL-2 to expand NK effector cells. Human PBMCs were co-cultured with WT, TKO, and 5GKO ipLDEC lines at effector:target (E:T) ratio of 10:1 for 2 hours, and harvested for fluorochrome-conjugated antibodies against CD45, CD3, CD56, CD107a cell surface marker staining. Human NK cell degranulation to porcine cell stimulation was measured by the percentage of CD107a-positive cells and surface expression of CD107a in the CD3-CD56+ cell population. There were no significant differences in either percentage or mean fluorescent intensity (MFI) of CD107a among WT, TKO, and 5GKO ipLDEC lines (FIG. 5A, 5B), suggesting that porcine cells lack inhibitory ligands and/or express activating ligands to trigger human NK cell activation.

### Methods

Characterization of Porcine Liver-Derived Endothelial Cells (ipLDEC).

Wild-type (WT) porcine liver-derived cells were cultured in media (α-MEM:EGM-MV 3:1) (Invitrogen/Lonza, Switzerland) supplemented with 10% FBS (Hyclone, Logan, UT), 10% horse serum (Invitrogen, Carlsbad, CA), 12 mM HEPES, and 1% pen/strep (Invitrogen), as described in previously¹⁷. Cells were stained with mouse anti-pig CD31-FITC (Bio-Rad Laboratories, Hercules, CA) and sheep anti-Von Willebrand Factor (VWF)-FITC (Abcam, Cambridge, MA). Isotype antibody was used as a control. pLDEC were treated with porcine IFN-7 (R&D Systems, Minneapolis, MN) at 10 ng/mL for 40 hours, SLA-I and SLA-II expression were examined by flow cytometric analysis with mouse anti-pig SLA-I-FITC antibody (Bio-Rad Laboratories) and mouse anti-pig SLA-II DR-FITC antibody (Bio-Rad Laboratories). Untreated pLDEC and isotype antibodies were used as controls.

Immortalization of pLDEC.

pLDEC were immortalized by SV40 T antigen and hTERT lentiviral treatment, according to the manufacturer's instruction (Applied Biological Materials, Richmond, BC, Canada). After immortalization, ipLDECs were kept in culture for over three months.

### Knockout of Multiple-Gene Encoding Xenoantigens.

In order to delete multiple porcine genes using CRISPR/Cas9, the gRNAs targeting GGTA1, CMAH, β4galNT2, SLA-I α chain, and B2M were designed and cloned to CRISPR/Cas9 vector pX330 and/or PX458, respectively. pX330-U6-Chimeric_BB-CBh-hSpCas9 and pSpCas9(BB)-2A-GFP (PX458) were gifts from Dr. Feng Zhang (Addgene plasmid #42230 and #48138). The sequences of gRNA target and gRNA oligos are listed in Table 1. Co-transfection of ipLDEC with multiple gRNAs targeting GGTA1, CMAH, and β4galNT2 genes was performed as previously described. Briefly, 106 of ipLDEC were mixed with 2 μgs of each plasmid and electroporated at 1300 V, 30 ms, 1 pulse using Neon Transfection System (Thermo Fisher Scientific, Waltham, MA). After 48 hours, transfected cells were enriched by flow sorting for GFP-positive cells, and sequentially selected for the absence of αGal and Sda, and reduction of Neu5Gc, by Dynabeads isolation and flow cytometry cell sorting.9,11,35 The reagents used for selection were Isolectin B4 (IB4)-biotin (Enzo Life Sciences, Farmingdale, NY), Dynabeads Biotin Binder (Thermo Fisher Scientific), Dolichos biflorus Agglutinin (DBA)-FITC (Vector Laboratories, Burlingame, CA), and chicken anti-Neu5Gc antibody-Alexa Fluor 488 (BioLegend, San Diego, CA). The mutation region of each gene was confirmed by PCR amplification and Sanger sequencing using specific primers (Table 2). Triple gene knockout (TKO; GGTA1/CMAH/β4galNT2) ipLDEC were generated.

TABLE 1
Sequences of gRNA target and gRNA oligos.

| | gRNA | |
|---|---|---|
| Gene | target | gRNA oligo |
| GGTA1 | GAGAAAATAATGAATGTCAA | 5′-CACCGAGAAAATAATGAATGTCAA-3′ |
| | (SEQ ID NO: 6) | (SEQ ID NO: 12) |
| | | 5′-AAACTTGACATTCATTATTTTCTC-3′ |
| | | (SEQ ID NO: 13) |
| CMAH | GAGTAAGGTACGTGATCTGT | 5′-CACCGAGTAAGGTACGTGATCTGT-3′ |
| | (SEQ ID NO: 7) | (SEQ ID NO: 14) |
| | | 5′-AAACACAGATCACGTACCTTACTC-3′ |
| | | (SEQ ID NO: 15) |
| β4galNT2 | CTGTATCGAGGAACACGCTT | 5′-CACCGTGTATCGAGGAACACGCTT-3′ |
| | (SEQ ID NO: 8) | (SEQ ID NO: 16) |
| | | 5′-AAACAAGCGTGTTCCTCGATACAC-3′ |
| | | (SEQ ID NO: 17) |
| B2M | CTCGTGGCCTTGGTCCTGCT | 5′-CACCGTCGTGGCCTTGGTCCTGCT-3′ |
| | (SEQ ID NO: 9) | (SEQ ID NO: 18) |
| | | 5′-AAACAGCAGGACCAAGGCCACGAC-3′ |
| | | (SEQ ID NO: 19) |
| SLA-1,2,3 | ATCATGTACGGCTGCGACGTG | 5′-CACCGTCATGTACGGCTGCGACGT-3′ |
| (1) | (SEQ ID NO: 10) | (SEQ ID NO: 20) |
| | | 5′-AAACACGTCGCAGCCGTACATGAC-3′ |
| | | (SEQ ID NO: 21) |
| SLA-1,2,3 | ACTATTGGGATGAGGAGACGC | 5′-CACCGCTATTGGGATGAGGAGACG-3′ |
| (2) | GG (SEQ ID NO: 11) | (SEQ ID NO: 22) |
| | | 5′-AAACCGTCTCCTCATCCCAATAGC-3′ |
| | | (SEQ ID NO: 23) |

TABLE 2
PCR primers used to amplify gRNA targeting regions

| Gene | PCR primers | Amplicon (bp) |
|---|---|---|
| GGTA1 | CCTTAGTATCCTTCCCAACCCAGAC (SEQ ID | 428 |
| | NO: 24) | |
| | GCTTTCTTTACGGTGTCAGTGAATCC (SEQ | |
| | ID NO: 25) | |
| CMAH | CTTGGAGGTGATTTGAGTTGGG (SEQ ID NO: | 458 |
| | 26) | |
| | CATTTTCTTCGGAGTTGAGGGC (SEQ ID NO: | |
| | 27) | |
| β4galNT2 | CGCAAGTGACCAGACATCGTTC (SEQ ID NO: | 530 |
| | 28) | |
| | AAAGCCACAGGAGGAGCCAG (SEQ ID NO: | |
| | 29) | |
| B2M | TCTTTCTAACCTGCTCGGGC (SEQ ID NO: 30) | 530 |
| | CGATCTGAAGCTTACCCGCA (SEQ ID NO: | |
| | 31) | |

Due to the high polymorphism of SLA-I α chain, several gRNAs targeting the common sequences of SLA-1, SLA-2, and SLA-3 multiple alleles were designed and tested for efficiency. TKO ipLDEC were transfected with two selected gRNAs targeting SLA-I α chains. After 48 hours, transfected cells were stained with mouse anti-SLA class I-FITC (Bio-Rad Laboratories) and SLA-I-deficient cells were selected by flow cytometry cell sorting. Then, this knockout cell line (GGTA1/CMAH/β4GalNT2 SLA-I α chain) was transfected with the gRNA targeting B2M gene. B2M mutant cells were selected by a single-clone screening approach using PCR and Sanger DNA sequencing. 5-gene knockout (5GKO; GGTA1/CMAH/β4GalNT2 SLA-I α chain B2M) ipLDECs were established.

### Human Antibody Binding to TKO and 5GKO Porcine Cells.

A human antibody binding assay was performed as previously described^{11,16}. Briefly, 2×10⁵ TKO or 5GKO ipLDECs were washed and incubated with 25% heat-inactivated human serum in EX-CELL 610-HSF Serum-Free Medium (Sigma, St. Louis, MO) with 0.1% sodium azide for 1 hour at 4° C. Cells were washed 3 times with EX-CELL, and stained with donkey anti-human IgG Alexa Fluor 488 (Jackson ImmunoResearch Laboratories, West Grove, PA) or donkey anti-human IgM Alexa Fluor 647 (Jackson ImmunoResearch Laboratories) for 30 minutes at 4° C. Cells were washed, fixed with 2% paraformaldehyde (PFA), and subsequently analyzed using a LSR2 flow cytometer (BD Biosciences, San Jose, CA). Flow data were analyzed using FlowJo v10 software (Tree Star, San Carlos, CA).

Human MNK cell degranulation. Commercially available buffy coats were acquired from Versiti Indiana Blood Center. Human peripheral blood mononuclear cells (PBMC) were isolated using Ficoll-Paque Plus (GE-Healthcare, Pittsburgh, PA) gradient centrifugation and cultured in RPMI1640 with 10% FBS, 1% pen/strep, and 20 ng/mL human IL-2 (BioLegend) at 37° C. in a CO2 incubator for 5 days. 5×104 WT, TKO, and 5GKO ipLDECs were plated in 48-well plates in duplicate or triplicate one day ahead of co-culture. 5×105 PBMC were added to porcine cells at an E:T ratio of 10:1, and co-cultured for 2 hours at 37° C. in a CO2 incubator. Then cells were collected and stained with fluorochrome-conjugated antibodies against human CD45, CD3, CD56, CD107a (BioLegend), and Invitrogen eBioscience Fixable Viability Dye eFluor 780 (Thermo Fisher Scientific). Stained cells were fixed with 2% PFA and subsequently analyzed using a LSR4 flow cytometer (BD Biosciences). After pre-gating on CD45+ live singlets, NK cell degranulation activity was assessed based on percentage and mean fluorescence intensity (MFI) of CD107a in a CD3-CD56+ cell population. Flow data were analyzed using FlowJo v10 software (Tree Star).

### Statistical Analysis.

Statistical analyses were performed using GraphPad Prism 8 software (GraphPad Software, San Diego, CA). Student's t-test was used to analyze the differences between two groups. One-way ANOVA, Tukey's multiple comparisons test was used to analyze the differences among multiple groups.

### REFERENCES

1 Ekser, B., Li, P. & Cooper, D. K. C. Xenotransplantation: past, present, and future. Curr Opin Organ Transplant 22, 513-521, doi:10.1097/MOT.0000000000000463 (2017).
2 Cooper, D. K., Ekser, B., Ramsoondar, J., Phelps, C. & Ayares, D. The role of genetically engineered pigs in xenotransplantation research. J Pathol 238, 288-299, doi:10.1002/path.4635 (2016).
3 Cooper, D. K. C. et al. Justification of specific genetic modifications in pigs for clinical organ xenotransplantation. Xenotransplantation 26, e12516, doi:10.1111/xen.12516 (2019).
4 Adams, A. B. et al. Xenoantigen Deletion and Chemical Immunosuppression Can Prolong Renal Xenograft Survival. Ann Surg 268, 564-573, doi:10.1097/SLA.0000000000002977 (2018).
5 Kim, S. C. et al. Long-term survival of pig-to-rhesus macaque renal xenografts is dependent on CD4 T cell depletion. Am J Transplant 19, 2174-2185, doi:10.1111/ajt.15329 (2019).
6 Langin, M. et al. Consistent success in life-supporting porcine cardiac xenotransplantation. Nature 564, 430-433, doi:10.1038/s41586-018-0765-z (2018).
7 Mohiuddin, M. M. et al. Chimeric 2C10R4 anti-CD40 antibody therapy is critical for long-term survival of GTKO.hCD46.hTBM pig-to-primate cardiac xenograft. Nat Commun 7, 11138, doi:10.1038/ncomms11138 (2016).
8 Li, P. et al. Efficient generation of genetically distinct pigs in a single pregnancy using multiplexed single-guide RNA and carbohydrate selection. Xenotransplantation 22, 20-31, doi:10.1111/xen.12131 (2015).
9 Estrada, J. L. et al. Evaluation of human and non-human primate antibody binding to pig cells lacking GGTA1/CMAH/beta4GalNT2 genes. Xenotransplantation 22, 194-202, doi:10.1111/xen.12161 (2015)
10 Fischer, K. et al. Viable pigs after simultaneous inactivation of porcine MHC class I and three xenoreactive antigen genes GGTA1, CMAH and B4GALNT2. Xenotransplantation 27, e12560, doi:10.1111/xen.12560 (2020)
11 Lutz, A. J. et al. Double knockout pigs deficient in N-glycolylneuraminic acid and galactose alpha-1,3-galactose reduce the humoral barrier to xenotransplantation. Xenotransplantation 20, 27-35, doi:10.1111/xen.12019 (2013).
12 Varki, A. Diversity in the sialic acids. Glycobiology 2, 25-40, doi:10.1093/glycob/2.1.25 (1992).
13 Li, Q. et al. Carbohydrate antigen expression and anti-pig antibodies in New World capuchin monkeys: Relevance to studies of xenotransplantation. Xenotransplantation 26, e12498, doi:10.1111/xen.12498 (2019).
14 Yamamoto, T. et al. Old World Monkeys are less than ideal transplantation models for testing pig organs lacking three carbohydrate antigens (Triple-Knockout). Sci Rep 10, 9771, doi:10.1038/s41598-020-66311-3 (2020).
15 Burdorf, L., Azimzadeh, A. M. & Pierson, R. N., 3rd. Progress and challenges in lung xenotransplantation: an update. Curr Opin Organ Transplant 23, 621-627, doi:10.1097/MOT.0000000000000582 (2018).
16 Martens, G. R. et al. Humoral Reactivity of Renal Transplant-Waitlisted Patients to Cells From GGTA1/CMAH/B4GalNT2, and SLA Class I Knockout Pigs. Transplantation 101, e86-e92, doi:10.1097/TP.0000000000001646 (2017).
17 Li, P., Estrada, J., Zhang, F., Waghmare, S. K. & Mir, B. Isolation, characterization, and nuclear reprogramming of cell lines derived from porcine adult liver and fat. Cell Reprogram 12, 599-607, doi:10.1089/cell.2010.0006 (2010).
18 Paris, L. L. et al. Reduced human platelet uptake by pig livers deficient in the asialoglycoprotein receptor 1 protein. Xenotransplantation 22, 203-210, doi:10.1111/xen.12164 (2015).
19 Martens, G. R. et al. HLA Class I-sensitized Renal Transplant Patients Have Antibody Binding to SLA Class I Epitopes. Transplantation 103, 1620-1629, doi:10.1097/TP.0000000000002739 (2019).
20 Diaz Varela, I., Sanchez Mozo, P., Centeno Cortes, A., Alonso Blanco, C. & Valdes Canedo, F. Cross-reactivity between swine leukocyte antigen and human anti-HLA-specific antibodies in sensitized patients awaiting renal transplantation. J Am Soc Nephrol 14, 2677-2683, doi:10.1097/01.asn.0000088723.07259.cf (2003).
21 Shimizu, A. et al. Acute humoral xenograft rejection: destruction of the microvascular capillary endothelium in pig-to-nonhuman primate renal grafts. Lab Invest 80, 815-830, doi:10.1038/labinvest.3780086 (2000).
22 Ladowski, J. M. et al. Swine Leukocyte Antigen Class II Is a Xenoantigen. Transplantation 102, 249-254, doi:10.1097/TP.0000000000001924 (2018).
23 Gao, H. et al. Human IL-6, IL-17, IL-1beta, and TNF-alpha differently regulate the expression of pro-inflammatory related genes, tissue factor, and swine leukocyte antigen class I in porcine aortic endothelial cells. Xenotransplantation 24, doi:10.1111/xen.12291 (2017).
24 Tran, P. D. et al. Porcine cells express more than one functional ligand for the human lymphocyte activating receptor NKG2D. Xenotransplantation 15, 321-332, doi:10.1111/j.1399-3089.2008.00489.x (2008).
25 Hein, R. et al. Triple (GGTA1, CMAH, B2M) modified pigs expressing an SLA class I (low) phenotype-Effects on immune status and susceptibility to human immune responses. Am J Transplant, doi:10.1111/ajt.15710 (2019).
26 Sake, H. J. et al. Possible detrimental effects of beta-2-microglobulin knockout in pigs. Xenotransplantation 26, e12525, doi:10.1111/xen.12525 (2019).
27 Inverardi, L. et al. Early recognition of a discordant xenogeneic organ by human circulating lymphocytes. J Immunol 149, 1416-1423 (1992).
28 Khalfoun, B. et al. Development of an ex vivo model of pig kidney perfused with human lymphocytes. Analysis of xenogeneic cellular reactions. Surgery 128, 447-457, doi:10.1067/msy.2000.107063 (2000).
29 Xu, H. et al. Prolonged discordant xenograft survival and delayed xenograft rejection in a pig-to-baboon orthotopic cardiac xenograft model. J Thorac Cardiovasc Surg 115, 1342-1349, doi:10.1016/50022-5223(98)70218-1 (1998).
30 Quan, D. et al. Identification, detection, and in vitro characterization of cynomolgus monkey natural killer cells in delayed xenograft rejection of hDAF transgenic porcine renal xenografts. Transplant Proc 32, 936-937, doi:10.1016/s0041-1345(00)01046-0 (2000).
31 Hadad, U., Martinez, O. & Krams, S. M. NK cells after transplantation: friend or foe. Immunol Res 58, 259-267, doi:10.1007/si2026-014-8493-4 (2014).
32 Sullivan, J. A., Oettinger, H. F., Sachs, D. H. & Edge, A. S. Analysis of polymorphism in porcine MHC class I genes: alterations in signals recognized by human cytotoxic lymphocytes. J Immunol 159, 2318-2326 (1997).
33 Kwiatkowski, P. et al. Induction of swine major histocompatibility complex class I molecules on porcine endothelium by tumor necrosis factor-alpha reduces lysis by human natural killer cells. Transplantation 67, 211-218, doi:10.1097/00007890-199901270-00005 (1999).
34 Alter, G., Malenfant, J. M. & Altfeld, M. CD107a as a functional marker for the identification of natural killer cell activity. J Immunol Methods 294, 15-22, doi:10.1016/j.jim.2004.08.008 (2004).
35 Li, P., Estrada, J. L., Burlak, C. & Tector, A. J. Biallelic knockout of the alpha-1,3 galactosyltransferase gene in porcine liver-derived cells using zinc finger nucleases. J Surg Res 181, e39-45, doi:10.1016/j.jss.2012.06.035 (2013).

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

What is claimed:

1. A genetically modified porcine cell comprising a knock-out mutation in each of genes GGTA, CMAH, β4galNT2, SLA-1, and B2M.

2. The porcine cell of claim 1, wherein the porcine cell is a porcine liver-derived endothelial cell (pLDEC).

3. The porcine cell of claim 2, wherein the porcine cell expresses CD31 and von Willebrand factor (VWF).

4. The porcine cell of claim 1, wherein the porcine cell exhibits reduced immunoreactivity to a human serum sample compared to a genetically modified porcine cell comprising knock-out mutations only in genes GGTA, CMAH, and β4galNT2.

5. The porcine cell of claim 1, wherein the porcine cell comprises one or more immortalization factor selected from SV40 T antigen, human telomerase (hTERT), simian virus 40 large T antigen, papillomavirus E6, papillomavirus E7, adenovirus E1A, Epstein-Barr virus, and human T-cell leukemia virus.

6. An organoid comprising the cell of claim 1.

7. A porcine tissue comprising the cell of claim 1

8. A pig comprising the cell of claim 1.

9. In vitro cultured porcine tissue comprising porcine cells with a knock-out mutation in each of genes GGTA, CMAH, β4galNT2, SLA-1, and B2M, optionally, wherein the tissue is suitable for transplantation.

10. The in vitro cultured porcine tissue of claim 9, wherein the porcine cells are porcine liver-derived endothelial cell (pLDEC).

11. The in vitro cultured porcine tissue of claim 9, wherein the tissue exhibits reduced immunoreactivity to a human serum sample, compared to in vitro cultured porcine tissue comprising cells with knock-out mutations only in GGTA, CMAH, and β4galNT2.

12. An animal comprising the cell of claim 1.

13. An animal comprising the in vitro cultured tissue of claim 9.

14. A method of testing genetically modified porcine cells for xenoreactivity comprising:
(a) providing genetically modified porcine cells comprising knock-out mutations in each of genes GGTA, CMAH, β4galNT2, SLA-1, and B2M,
(b) incubating human cytotoxic cells in the presence and absence of the genetically modified porcine cells of (a);
(c) detecting a level of activation or degranulation of the human cytotoxic cells in the presence and absence of the genetically modified porcine cells of (a);
wherein, if the level of activation or degranulation of the human cytotoxic cells is increased in the presence of the genetically modified porcine cells as compared to the absence of the genetically modified porcine cells, then the genetically modified porcine cells are xenoreactive.

15. The method of claim 14, wherein the cytotoxic cells comprise natural killer (NK) cells.

16. The method of claim 14, wherein the cytotoxic cells comprise T cells.

17. The method of claim 16, wherein the T cells are CD8⁺ T cells.

18. The method of claim 16, wherein the T cells are CD4⁺ T cells.

19. The method of claim 14, wherein detecting a level of activation or degranulation of the human cytotoxic cells comprises detecting one or more of: interferon gamma (IFN-γ) production, granzyme b expression, surface CD107a, or tumor necrosis factor alpha (TNFα) production.

20. The method of claim 14, wherein detecting comprises flow cytometry.


SEQUENCE LISTING (39 sequences)

SEQ ID NO: 1 - protein, 371 aa, Sus scrofa
MNVKGRVVLSMLLVSTVMVVFWEYINSPEGSLFWIYQSKNPEVGSSAQRGWWFPSWFNNGTHSYHEEEDAIGNEKEQRKEDNRGELPLVDWFNPEKRPEVVTITRWKAPVVWEGTYNRAVLDNYYAKQKITVGLTVFAVGRYIEHYLEEFLISANTYFMVGHKVIFYIMVDDISRMPLIELGPLRSFKVFEIKSEKRWQDISMMRMKTIGEHILAHIQHEVDFLFCMDVDQVFQNNFGVETLGQSVAQLQAWWYKAHPDEFTYERRKESAAYIPFGQGDFYYHAAIFGGTPTQVLNITQECFKGILQDKENDIEAEWHDESHLNKYFLLNKPTKILSPEYCWDYHIGMSVDIRIVKIAWQKKEYNLVRNNI

SEQ ID NO: 2 - protein, 577 aa, Sus scrofa
MSSIEQTTEILLCLSPAEAANLKEGINFVRNKSTGKDYILFKNKSRLKACKNMCKHQGGLFIKDIEDLNGRSVKCTKHNWKLDVSSMKYINPPGSFCQDELVVEKDEENGVLLLELNPPNPWDSEPRSPEDLAFGEVQITYLTHACMDLKLGDKRMVFDPWLIGPAFARGWWLLHEPPSDWLERLSRADLIYISHMHSDHLSYPTLKKLAERRPDVPIYVGNTERPVFWNLNQSGVQLTNINVVPFGIWQQVDKNLRFMILMDGVHPEMDTCIIVEYKGHKILNTVDCTRPNGGRLPMKVALMMSDFAGGASGFPMTFSGGKFTEEWKAQFIKTERKKLLNYKARLVKDLQPRIYCPFAGYFVESHPADKYIKETNIKNDPNELNNLIKKNSEVVTWTPRPGATLDLGRMLKDPTDSKGIVEPPEGTKIYKDSWDFGPYLNILNAAIGDEIFRHSSWIKEYFTWAGFKDYNLVVRMIETDEDFSPLPGGYDYLVDFLDLSFPKERPSREHPYEEIRSRVDVIRHVVKNGLLWDDLYIGFQTRLQRDPDIYHHLFWNHFQIKLPLTPPDWKSFLMCSG

SEQ ID NO: 3 - protein, 566 aa, Sus scrofa
MGSAGFSVGKFHVEVASRGRECVSGTPECGNRLGSAGFGALCLELRGADPAWGPFAAHGRSRRQGSRFLWLLKILVIILVLGIVGFMFGSMFLQAVFSSPKPELPSPAPGVQKLKLLPEERLRNLFSYDGIWLFPKNQCKCEANKEQGGYNFQDAYGQSDLPAVKARRQAEFEHFQRREGLPRPLPLLVQPNLPFGYPVHGVEVMPLHTVPIPGLQFEGPDAPVYEVTLTASLGTLNTLADVPDSVVQGRGQKQLIISTSDRKLLKFILQHVTYTSTGYQHQKVDIVSLESRSSVAKFPVTIRHPVIPKLYDPGPERKLRNLVTIATKTFLRPHKLMIMLRSIREYYPDLTVIVADDSQKPLEIKDNHVEYYTMPFGKGWFAGRNLAISQVTTKYVLWVDDDFLFNEETKIEVLVDVLEKTELDVVGGSVLGNVFQFKLLLEQSENGACLHKRMGFFQPLDGFPSCVVTSGVVNFFLAHTERLQRVGFDPRLQRVAHSEFFIDGLGTLLVGSCPEVIIGHQSRSPVVDSELAALEKTYNTYRSNTLTRVQFKLALHYFKNHLQCAA

SEQ ID NO: 4 - protein, 118 aa, Sus scrofa
MAPLVALVLLGLLSLSGLDAVARPPKVQVYSRHPAENGKPNYLNCYVSGFHPPQIEIDLLKNGEKMNAEQSDLSFSKDWSFYLLVHTEFTPNAVDQYSCRVKHVTLDKPKIVKWDRDH

SEQ ID NO: 5 - protein, 361 aa, Sus scrofa
MGPGALFLLLSGTLALTGTQAGPHSLSYFYTAVSRPDRGDSRFIAVGYVDDTQFVRFDNYAPNPRMEPRVPWIQQEGQEYWDRETRNVKETAQTYGVGLNTLRGYYNQSEAGSHTLQSMYGCYLGPDGLLLHGYRQDAYDGADYIALNEDLRSWTAADMAAQITKRKWEAADEAERRRSYLQGLCVESLRRYLEMGKDTLQRAEPPKTHVTRHPSSDLGVTLRCWALGFYPKEISLTWQREGQDQSQDMELVETRPSGDGTFQKWAALVVPPGEEQSYTCHVQHEGLQEPLTLRWDPAQPPVPIVGIIVGLVLVLVAGAMVAGVVIWRKTRSGEKGGSYTQAAGSDSDQGSDVSLTKDPRV

SEQ ID NO: 6 - DNA, 20 bp, Artificial Sequence
  Synthetic
gagaaaataatgaatgtcaa

SEQ ID NO: 7 - DNA, 20 bp, Artificial Sequence
  Synthetic
gagtaaggtacgtgatctgt

SEQ ID NO: 8 - DNA, 20 bp, Artificial Sequence
  Synthetic
gagtaaggtacgtgatctgt

SEQ ID NO: 9 - DNA, 20 bp, Artificial Sequence
  Synthetic
ctcgtggccttggtcctgct

SEQ ID NO: 10 - DNA, 21 bp, Artificial Sequence
  Synthetic
atcatgtacggctgcgacgtg

SEQ ID NO: 11 - DNA, 23 bp, Artificial Sequence
  Synthetic
actattgggatgaggagacgcgg

SEQ ID NO: 12 - DNA, 24 bp, Artificial Sequence
  Synthetic
caccgagaaaataatgaatgtcaa

SEQ ID NO: 13 - DNA, 24 bp, Artificial Sequence
  Synthetic
aaacttgacattcattattttctc

SEQ ID NO: 14 - DNA, 24 bp, Artificial Sequence
  Synthetic
caccgagtaaggtacgtgatctgt

SEQ ID NO: 15 - DNA, 24 bp, Artificial Sequence
  Synthetic
aaacacagatcacgtaccttactc

SEQ ID NO: 16 - DNA, 24 bp, Artificial Sequence
  Synthetic
caccgtgtatcgaggaacacgctt

SEQ ID NO: 17 - DNA, 24 bp, Artificial Sequence
  Synthetic
aaacaagcgtgttcctcgatacac

SEQ ID NO: 18 - DNA, 24 bp, Artificial Sequence
  Synthetic
caccgtcgtggccttggtcctgct

SEQ ID NO: 19 - DNA, 24 bp, Artificial Sequence
  Synthetic
aaacagcaggaccaaggccacgac

SEQ ID NO: 20 - DNA, 24 bp, Artificial Sequence
  Synthetic
caccgtcatgtacggctgcgacgt

SEQ ID NO: 21 - DNA, 24 bp, Artificial Sequence
  Synthetic
aaacacgtcgcagccgtacatgac

SEQ ID NO: 22 - DNA, 24 bp, Artificial Sequence
  Synthetic
caccgctattgggatgaggagacg

SEQ ID NO: 23 - DNA, 24 bp, Artificial Sequence
  Synthetic
aaaccgtctcctcatcccaatagc

SEQ ID NO: 24 - DNA, 25 bp, Artificial Sequence
  Synthetic
ccttagtatccttcccaacccagac

SEQ ID NO: 25 - DNA, 26 bp, Artificial Sequence
  Synthetic
gctttctttacggtgtcagtgaatcc

SEQ ID NO: 26 - DNA, 22 bp, Artificial Sequence
  Synthetic
cttggaggtgatttgagttggg

SEQ ID NO: 27 - DNA, 22 bp, Artificial Sequence
  Synthetic
cattttcttcggagttgagggc

SEQ ID NO: 28 - DNA, 22 bp, Artificial Sequence
  Synthetic
cgcaagtgaccagacatcgttc

SEQ ID NO: 29 - DNA, 20 bp, Artificial Sequence
  Synthetic
aaagccacaggaggagccag

SEQ ID NO: 30 - DNA, 20 bp, Artificial Sequence
  Synthetic
tctttctaacctgctcgggc

SEQ ID NO: 31 - DNA, 20 bp, Artificial Sequence
  Synthetic
cgatctgaagcttacccgca

SEQ ID NO: 32 - DNA, 23 bp, Artificial Sequence
  Synthetic
gagaaaataatgaatgtcaaagg

SEQ ID NO: 33 - DNA, 22 bp, Artificial Sequence
  Synthetic
  misc_feature (1)..(22): n= a, c, t, or g
gagaaataatnnannnnnagng

SEQ ID NO: 34 - DNA, 26 bp, Artificial Sequence
  Synthetic
gagtaaggtacgtgatctgttggaag

SEQ ID NO: 35 - DNA, 14 bp, Artificial Sequence
  Synthetic
gagtaaggtacgtg

SEQ ID NO: 36 - DNA, 23 bp, Artificial Sequence
  Synthetic
ctgtatcgaggaacacgcttcgg

SEQ ID NO: 37 - DNA, 23 bp, Artificial Sequence
  Synthetic
  misc_feature (1)..(23): n= a, c, t, or g
ctgtatcgaggaacacnnnnnnn

SEQ ID NO: 38 - DNA, 27 bp, Artificial Sequence
  Synthetic
ctggtggccttggtcctgctttggctg

SEQ ID NO: 39 - DNA, 13 bp, Artificial Sequence
  Synthetic
ctcgtggccttgg